(19) United States
(12) Patent Application Publication
(10) Pub. No.: US 2004/0250813 A1
(43) Pub. Date: Dec. 16, 2004

(54) Portable digital anesthesia apparatus

(76) Inventors: Terry Michael Post, Belmont, NY (US); Dell Parker Baker JR., Belmont, NY (US); Donna Sue Clarke, Belmont, NY (US)

Correspondence Address: Terry M. Post, 5762 Watson Rd., Belmont, NY, 14813, US

(21) Appl. No.: 10/368,313

(22) Filed: Jun. 16, 2003

(51) Int. Cl. A61M 15/00

(52) U.S. Cl. 128/203.12

(57) ABSTRACT

The present invention solves two main problems inherent to anesthesia design and delivery. By controlling the evaporation of each drop of the liquid agent by providing the required heat at the point of evaporation, an extremely accurate delivery of vaporized anesthestic can be delivered safely to the patient and by using an inexpensive microcomputer, peltier junction or heating element and a liquid tight solenoid or a position controllable stepper motor, the whole machine is much easier and less expensive to construct. This coupled with the simple design of the body being cast in aluminum urethane, the present invention has substantially reduced the cost of manufacture while actually increasing the accuracy of it's output.

### 1. FIELD OF THE INVENTION

The present invention relates to a portable digitally controlled anesthesia apparatus used for administering general inhalational anesthestics to humans and other animals.

### 2. BACKGROUND OF THE INVENTION

General gas anesthesia involves the controlled release of evaporated anesthestic agents to the patient. Before the present invention this control was limited by the flow dynamics of oxygen either through the liquid agent or over the surface. The actual percent by volume of the evaporated agent depended upon the ambient conditions of both temperature and barometric pressure, minimal changes in either one results in a substantial difference in the output to the patient.

This sensitivity has forced the makers of anesthesia devices to build heavy, complex vaporizers with mechanical countermeasures to offset local changes in an attempt to deliver safe anesthesia. There is a general need and desire to posses a portable machine capable of accurate use in a field or remote location regardless of changes in both temperature and barometric pressure.

### SUMMARY OF THE INVENTION

The present invention provides a portable, digitally controlled, multiagent anesthesia apparatus, comprising:

It is an aspect of the present invention to provide a portable anesthesia apparatus.

It is another aspect of the present invention to provide an anesthestic apparatus that is durable and reliable.

It is another aspect of the present invention to provide an anesthetic apparatus capable of safe use in changing ambient conditions.

It is another aspect of the present invention to provide an anesthesia apparatus whose construction is simple and inexpensive.

It is another aspect of the present invention to provide the calibrated amount of heat at the point of evaporation for each discrete drop of liquid anesthestic agent common in the art.

It is another aspect of the present invention to provide a means by which a liquid tight seal can be obtained by casting a prepared rotatable shaft.

It is another aspect of the present invention to provide an anesthesia apparatus cast in one piece from aluminum urethane enveloping all piping and sensors.

It is another aspect of the present invention to provide an anesthesia apparatus employing a single reservoir qualified to hold all of the known anesthetic agents.

These and other apsects of the present invention are achieved herein.

### BRIEF DESCRIPTION OF THE DRAWINGS

The present invention is illustrated by the embodiments shown in the following drawings:

FIG. 1 Is an orthogonal view of the single cavity casting mold containing the piping and fixturing.

FIG. 7 Is an orthogonal rendered view of the single cavity casting mold with piping and fixtures in pre-casting position.

FIG. 12 Is an orthogonal view of the exploded piping and fixturing showing discrete components.

FIG. 26 Is an orthogonal view of piping and fixturing in the post casting phase showing the final relative position of same.

FIG. 38 Is an orthogonal view of piping and fixturing in the post casting phase showing the final relative position of same from the opposite side.

FIG. 53 Is an orthogonal view of the separately cast oxygen flow control apparatus with hidden lines shown as dashed.

FIG. 64 is an orthogonal view of the agent flow control stem with hidden lines dashed.

FIG. 70 Is an orthogonal view of the separately cast oxygen flow control apparatus with draft rendering showing exit port of the apparatus.

FIG. 78 Is a rendered orthogonal view of the final cast apparatus.

FIG. 79 is a schematic representation of the sensor systems the microcomputer and the heat supplier as well as the liquid agent control system.

### DETAILED DESCRIPTION OF THE PREFERRED EMBODIMENTS

The present invention relates to a portable multiagent digitally controlled anesthetic apparatus for evaporating any of the known in the art liquid anesthestic agents into a carrier stream of oxygen and can then be safely delivered to the surgical patient in a percentage of volume control.

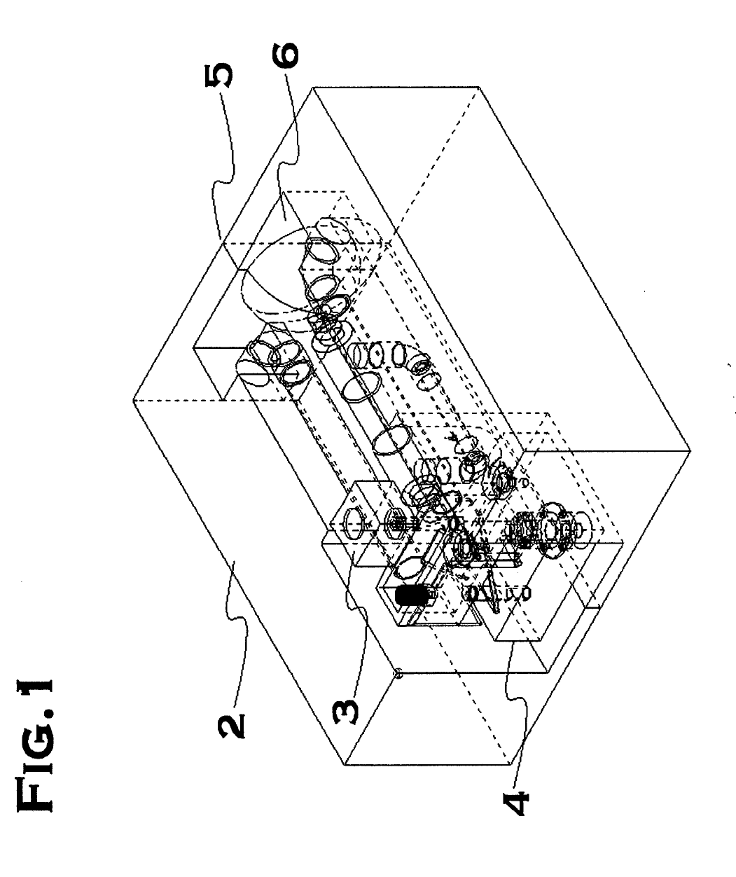

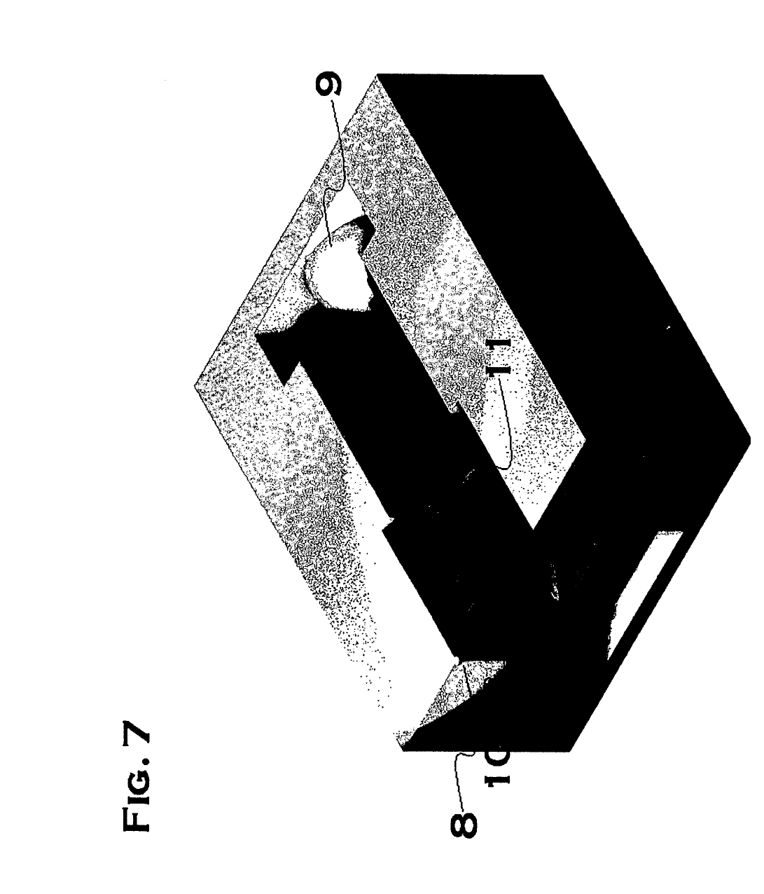

FIG. 1 shows an orthogonal draft view of a single cavity mold wherein the piping And fixturing are contained in the casting position. FIG. 2 defines one of two halves of the mold, made from casting in cement or any other material capable of holding aluminum urethane. FIG. 3 shows the relative position of the common in the art stepper motor. FIG. 4 depicts a plastic box insert creating a preformed cavity for the control systems. FIG. 5 shows the parting line between the two halves of the single cavity mold. FIG. 6 defines the fill area used in the pouring of the liquid aluminum urethane.

FIG. 7 is a rendered orthogonal view of the single cavity mold containing the piping and fixturing. FIG. 8 approximates the fill to level indication. At this level, the liquid aluminum urethane will set up with the piping and the fixturing in the appropriate positions for reliable use as well as user access. FIG. 9 shows the negative impression of the fill trough, once cast this will provide the large opening which drains into the main liquid reservoir seen in FIG. 17. FIG. 10 shows the threaded oxygen connector through which a constant supply of 50 psi of medical grade oxygen is attached. FIG. 11 defines the position of the peltier junction inside of it's closed container FIG. 21.

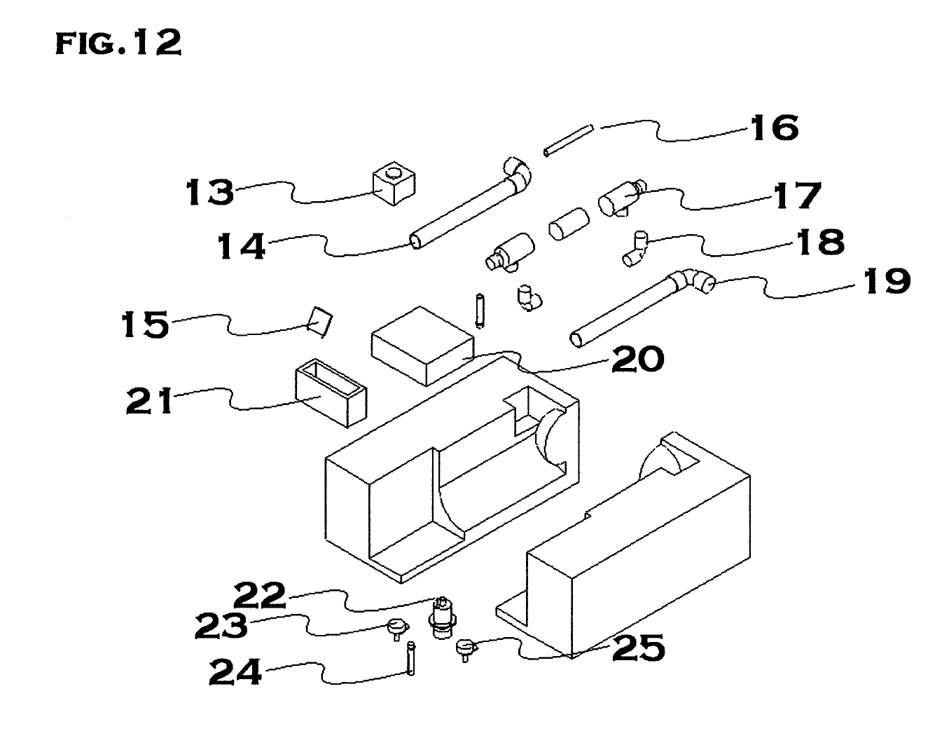

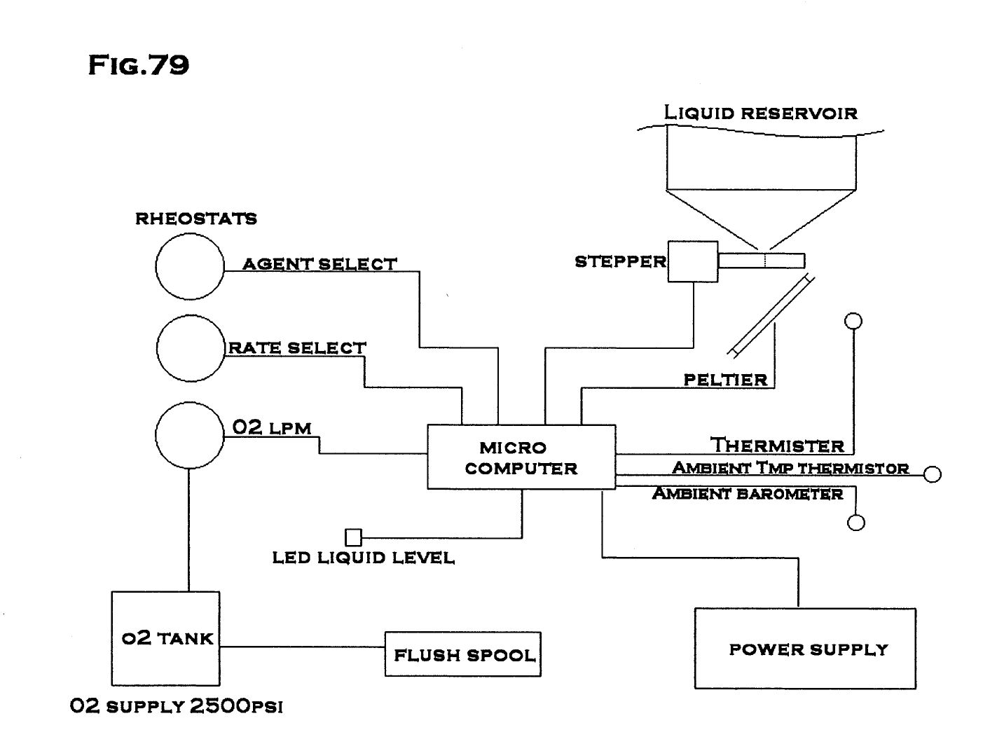

FIG. 12 further shows an exploded draft of the piping and fixturing. FIG. 13 shows the common in the art stepper motor. FIG. 14 and FIG. 19 are identical copper piping sections composed of a straight section attached to a 90 degree elbow. These comprise the inlet and outlet paths through which the carrier gas is joined with the breathing stream. FIG. 15 is a common in the art peltier junction which when in functional position rests at a 45 degree angle below the outlet port below the liquid reservoir seen in FIG. 17. As the liquid drop is isolated by the stepper's single complete rotation, it falls against the alumina surface of the peltier junction whose temperature can be controlled by the external computer. seen in FIG. 79.

FIG. 12 further defines the cluster of control systems {FIGS. 22,23,24 & 25} which are affixed inside of FIG. 20 before casting with aluminum urethane. FIG. 16 shows a borosilca glass tube which when positioned inside of two common in the art copper elbow fittings {FIG. 18}, can be cast producing a liquid tight sight glass when seen from the opposite side.

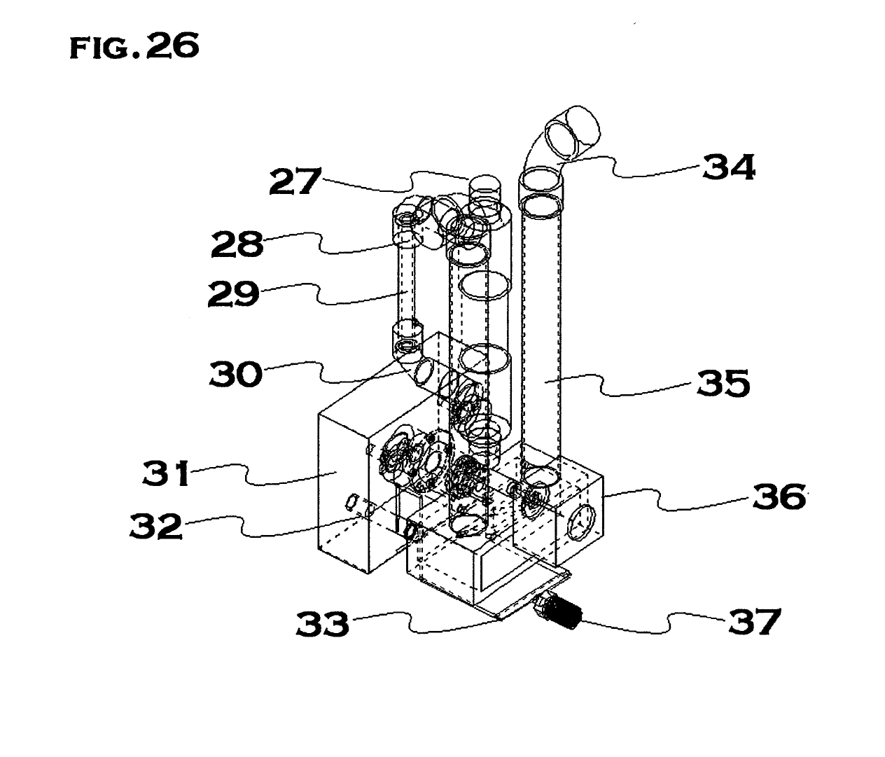

FIG. 26 depicts the positioned fixturing in an orthogonal view with hidden lines dashed. FIG. 27 shows the filling port which is cast up against the fill tough as seen in FIG. 9. FIG. 28 is the borosilica glass tube in the casting position. The tube is located with a durable adhesive compatible with the liquid agents. The tube is also coated with a silicon caulk which is removed after the casting and curing of the apparatus providing a clean view of the glass during operation. FIG. 30 shows the common in the art copper elbows which hold the sight glass in position and act as the liquid conduit for the agent, it reaches equilibrium and accurately displays the level of the agent in the main reservoir FIG. 17. FIG. 31 shows the control box in position with the oxygen flow control, agent control optical encoder and the agent selector optical encoder. The flush spool is defined in FIG. 32, FIG. 33 shows the 50 psi oxygen piping created by casting a lubricated monofliliment string connecting the inlet port {FIG. 37} as well as the oxygen flow control system {FIG. 22}, after the casting has cured the monofiliment line is removed leaving an internal conduit for the 50 psi of oxygen.

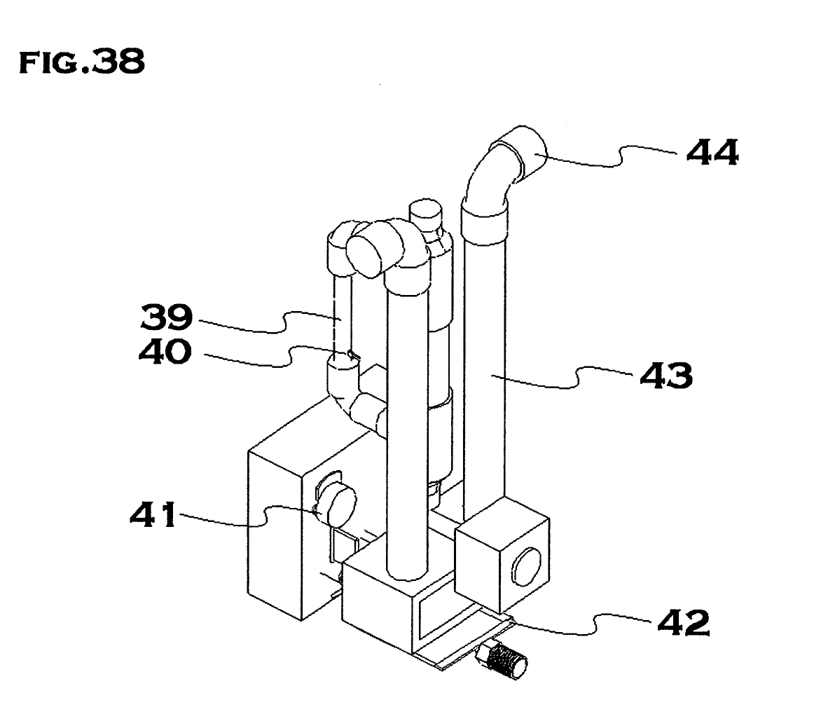

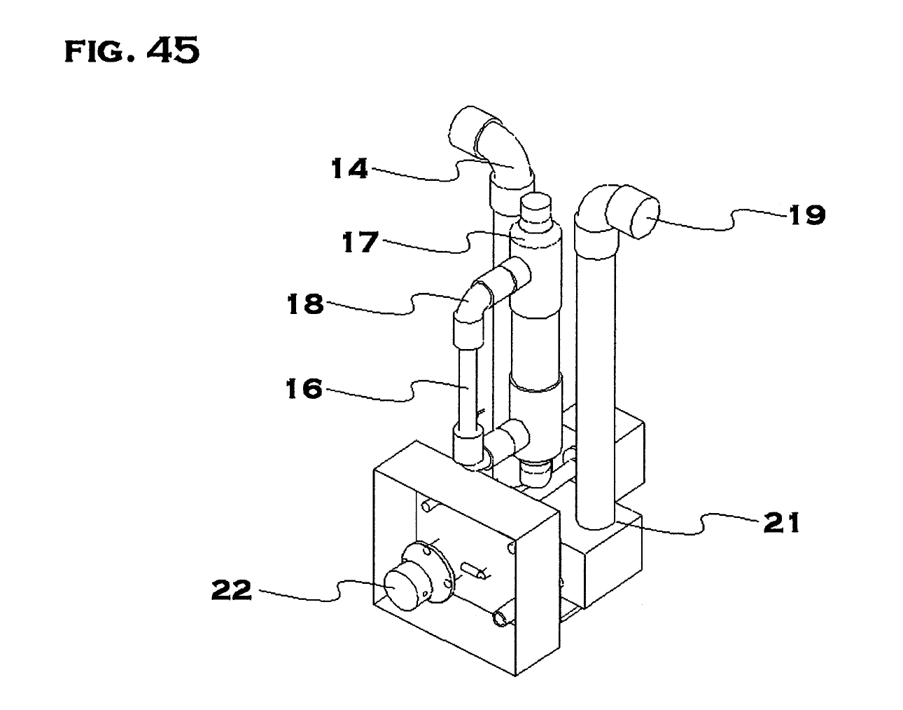

FIG. 38 shows a rendered orthogonal view of the piping and fixturing where FIG. 40 denotes the location of the LED lamp which via the microcomputer, lights the sight glass from behind giving a better view of the liquid level of the agent. FIG. 41 is the same as FIG. 25 with the difference being only the rendered aspect of the rheostat as seen from behind. FIG. 42 is a rendered view of the 50 psi oxygen conduit created using the monofiliment line described above. FIGS. 43 and 44 are rendered views of the common in the art copper tubing and elbows used to create the main inlet and outlet for the anesthesia apparatus.

FIG. 45 is the assembled view of the piping and fixturing with corresponding figure numbers from the exploded view FIG. 12.

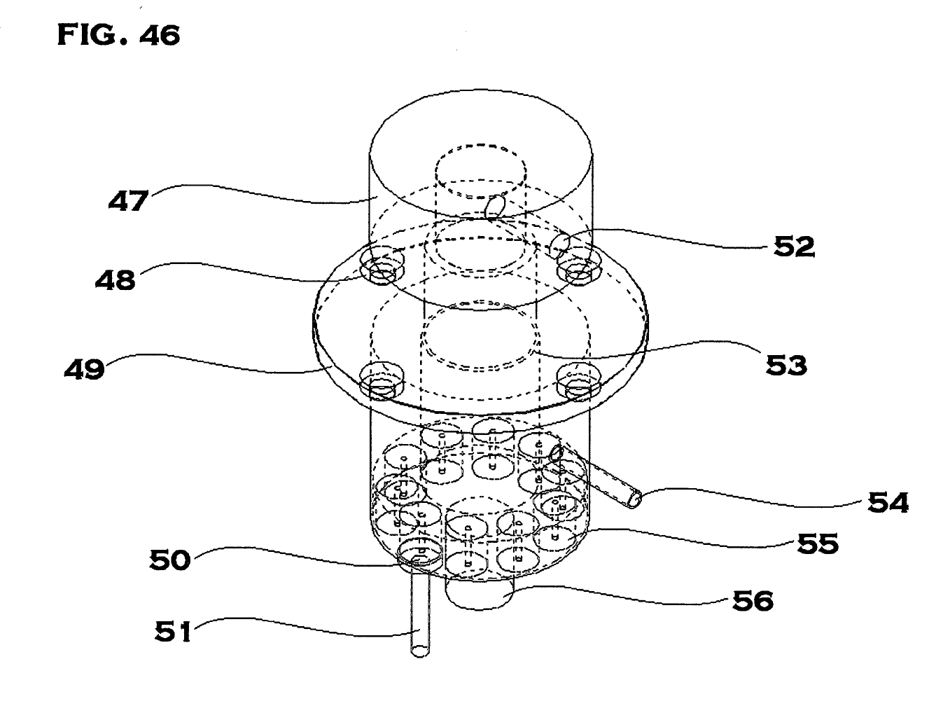

FIG. 46 shows the oxygen flow control system. FIG. 47 is the user control knob held in place by a set screw {FIG. 52}. The material of the assembly can be any light plastic, it's strength lies in the casting. A lubricated monofiliment line is fed through the plastic tube FIG. 51 from the 50 psi oxygen inlet {FIG. 37}, once the casting is cured the monofiliment line is removed and the patent conduit is left. FIG. 50 is the point at which the 50 psi oxygen presents to one of 11 small cast cylinders with graduated hole sizes thus providing a known flow rate by rotating the knob {FIG. 47}. FIG. 48 shows one of four bolt holes for securing the brass plate {FIG. 49} to the secondarily cast housing for the control systems FIG. 31. It has been found that when the lubricated bolts are cast in position, they can be removed leaving the threads in place allowing further work to be done inside of the control box FIG. 31. FIG. 55 shows one of 11 small aluminum urethane cast cylinders that are cast with a lubricated wire whose outside diameter will reduce the flow of oxygen as a function of diameter. FIG. 56 is the rotating seat that holds the oxygen flow control system in place, it too is lubricated prior to casting which later can be rotated after the casting has cured creating a tight smooth hold for the rotation of the oxygen flow control apparatus. FIG. 53 is the rubber “O” ring used to contain the graduated oxygen flow with out leakage during rotation of knob FIG. 47.

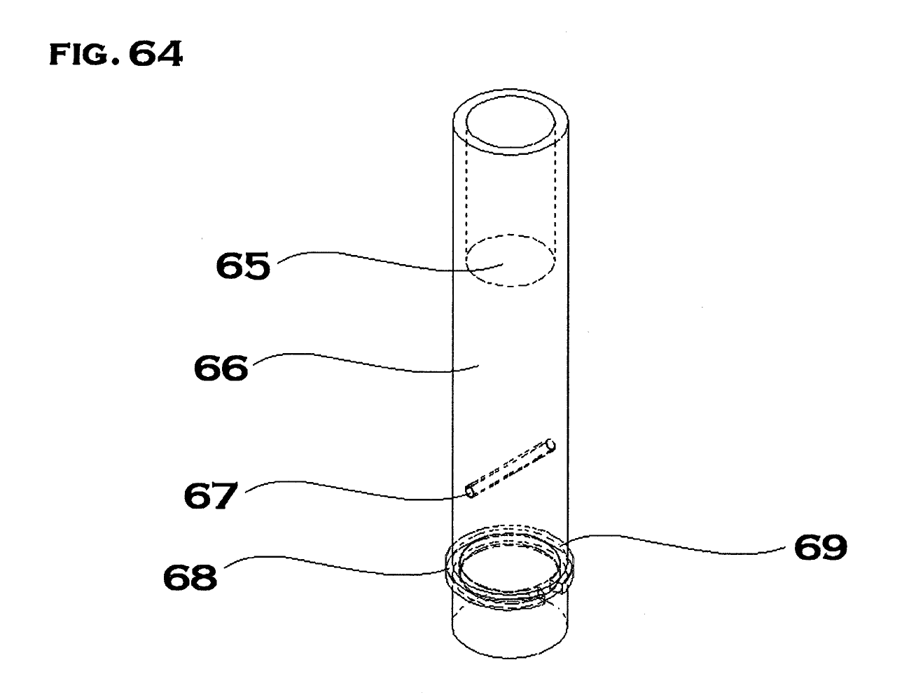

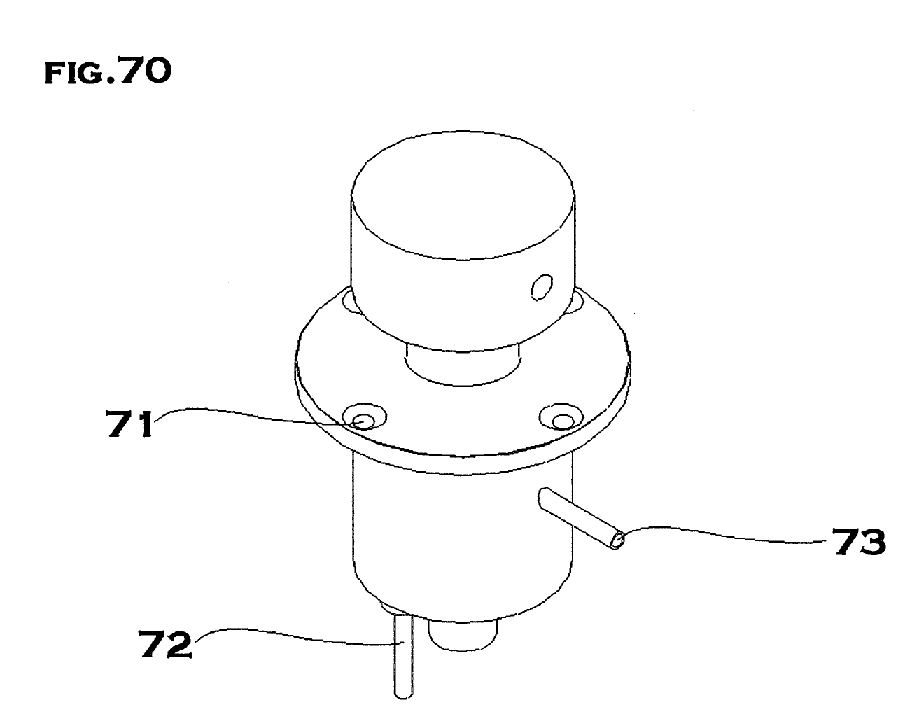

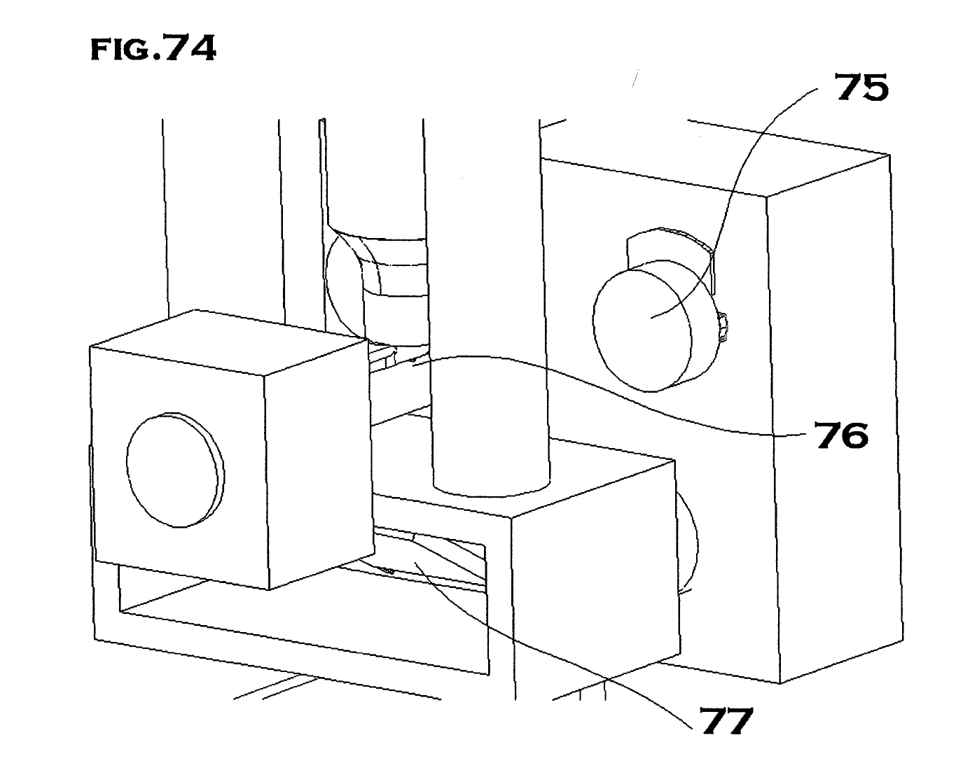

FIG. 64 is the flush control spool in orthogonal view. Constructed from cast aluminum urethane in a paper tube, FIG. 67 shows the final conduit created from a lubricated wire inserted through the paper tube during casting. Once removed, the wire leaves a conduit that is again threaded with a lubricated monofiliment line and the whole spool is cast in position. The monofiliment line is removed and the spool now when rotated allows an air tight seal when rotated in the off position. In the on position, the 50 psi of oxygen will provide the operator with a controllable flush mechanism. FIG. 68 and FIG. 69 show the slip ring and groove which holds the spool in place.

What is claimed is;

1. A portable digital anesthesia apparatus, comprising:
A single reservoir defined by a cavity made from known in the art copper tubing for containing any of the common use anesthetic agents.
A known in the art microcomputer that is programmed to read the temperature of a peltier junction used to provide the appropriate amount of heat for the rate of evaporation as defined by the user.

2. A portable digital anesthesia apparatus, comprising:
A body that is cast in place with all internals located in position of function using an aluminum urethane mixture that can be colored and polished.

3. A portable digital anesthesia apparatus, comprising:
A known in the art microcomputer that is programmed to allow electric current to flow to a peltier junction by using a known in the art Darlington array current driver chip in proportion to the user setting for delivery of the percent by volume of evaporated anesthetic agent as a dependant function of the flow rate of oxygen.

4. A portable digital anesthesia apparatus, comprising:
A borosilicate glass observation tube to adjudge remaining volume of liquid agent.

5. A portable digital anesthesia apparatus, comprising:
A known in the art microcomputer that is programmed to compute from a look up table, the controlling amounts of heat energy needed to completely evaporate a single drop of known anesthetic agent. As well as the concomitant heat required for numerous drops per second for extreme large tidal volume uses.

6. A portable digital anesthesia apparatus, comprising:
A known in the art microcomputer that is programmed to sense a known in the art barometric sensor to compute the partial pressure of the anesthetic agent as a function of oxygen flow as defined by a knob selector for the user input.

7. A portable digital anesthesia apparatus, comprising:
System of internal conduits for liquid and pressurized gases wherein a lubricated rod or string is positioned in a cavity that is filled with a liquid aluminum urethane mixture. When cured, the rod or string can be removed leaving an internal conduit of an exactly reproducible diameter as well as the ability to create said conduits using nonlinear rods or strings, that is uneven or not straight. This method allows a complex system of conduits to be manufactured easily and cheaply with engineered precision.

8. A portable digital anesthesia apparatus, comprising:
A large open fill port with a liquid tight cap which when cast according to claim 7 will easily create a threaded liquid tight cap for the anesthetic agent.

9. A portable digital anesthesia apparatus, comprising:
A system of mounting bolts and attachment screws using a template of threaded rods that can be cast in the position of function and location wherein the rods are lubricated and can be unscrewed from the aluminum urethane once cured leaving a very high tolerance location and threads for mounting hardware.

10. A portable digital anesthesia apparatus, comprising:
A known in the art surface mount 2,400 baud modem chip allowing external interface for long distance control of the apparatus by a qualified user over the internet using known in the art telecommunications.

11. A portable digital anesthesia apparatus, comprising:
An internal piping structure which allows operation in either direction that is either port can be used as the patient delivery port of the evaporated agent-oxygen combination. This function allows left or right hand operation as well as “draw over” function for use without a pressurized oxygen carrier supply.

12. A portable digital anesthesia apparatus, comprising:
In accordance with claim 11, by use of a simple one way check valve, the apparatus can be safely used in the absence of a supply of pressurized oxygen or other gas. By means of the known in the art microcomputer being programmed to detect the tidal flow of the patient's breathing using a known in the art gas flow transducer.

13. A portable digital anesthesia apparatus, comprising:
A computer controlled flow of single reproducible drops of anesthetic agent that flows onto the surface of a known in the art peltier junction which delivers the heat required for the computed rate of evaporation as a function of the detected ambient barometric pressure and the known rate of evaporation of each known agent.

14. A portable digital anesthesia apparatus, comprising:
A cast oxygen flow control apparatus using a rotar with revealing holes of increasing diameter wherein each of the holes are performed using the method described in claim 7. Once cured, the template rods whose outside diameter is equal to the computed diameter for oxygen at STP can be removed leaving exactly reproducible holes that will step down a 50 p.s.i. supply of oxygen into incremental flow rates.

15. A portable digital anesthesia apparatus, comprising:
A rugged aluminum urethane cast body with a resulting machine that is both shock proof and water proof

16. A portable digital anesthesia apparatus, comprising:
A resolution of accuracy in the present design of 65,000 to 1 in the control of the evaporation of each discrete drop of agent.
